(19) United States
(12) Patent Application Publication
(10) Pub. No.: US 2012/0014926 A1
(43) Pub. Date: Jan. 19, 2012

(54) Activation of Precursor Cells for Cell Therapy

(76) Inventor: Hong Yu, Maimi, FL (US)

(21) Appl. No.: 12/972,646

(22) Filed: Dec. 20, 2010

Related U.S. Application Data
(60) Provisional application No. 61/284,416, filed on Dec. 22, 2009.

(51) Int. Cl. A61K 35/12; C12N 5/071; C12N 5/07

(52) U.S. Cl. 424/93.7; 435/375; 435/352; 435/366

(57) ABSTRACT

This invention relates to a method and compositions for activation of precursor cells via upregulation of CXCR4 surface receptor expression, to give the activated cells a better homing capability and higher viability. The method for activation involves incubation of the target cells with an isotonic activation solution containing an activation-effective amount of calcium. Such activated cells have a better ability to engraft into target tissues to execute the therapeutic function.

This application claims the benefit of the priority of U.S. provisional application for patent 61/284,416, which is incorporated herein by reference in its entirety.

### FIELD OF THE INVENTION

This invention relates to methods and reagents useful for increasing the amount of CXCR4 receptor on cells, in order to activate the cells and thereby increase the engraftment of said cells into target tissues for cell-based therapy.

### BACKGROUND

Cell-based therapies have been used for treatment of different diseases, such as cardiovascular diseases^{1,2} (e.g. atherosclerosis, myocardial infarction, limb ischemia, and stroke), diabetes³, spinal cord injury and other neural diseases⁴, eye diseases⁵, immunological diseases and blood disorders⁶, and cancer⁷. For example, injection of bone marrow (BM)-derived^{1,8,9} or peripheral blood-derived¹⁰⁻¹³ mononuclear cells (MNC) into ischemic muscle has been shown to promote angiogenesis through the formation of new vascular collaterals that bypass the occluded arteries responsible for the ischemia. BM-derived progenitor cells participate in vascular repair process by engrafting onto the injured vascular sites and differentiating into endothelial cells¹⁴⁻¹⁹ or perivascular cells²⁰ that provide physical support and secrete signaling proteins and structural enzymes enabling the angiogenesis process. Laboratory and clinical studies have shown that implantation of BM derived cells (BMC) into ischemic limbs improves oxygen tension via collateral vessel formation. Kalka et al first demonstrated that transplantation of culture-expanded endothelial progenitor cells (EPC) successfully promoted neovascularization of the ischemic hindlimb.²¹ BM-MNC implantation into animal ischemic limbs^{22,23} or myocardium²⁴ promoted collateral vessel formation with incorporation of EPC into new capillaries. Cell-based neovascularization has also been indirectly achieved by mobilizing MNC from the BM with cytokines and chemokines such as VEGF, G-CSF, and SDF-1.^{19,25-28}

Another example of cell therapy is blood and marrow stem cell transplantation which has been widely used to replaces a person's abnormal stem cells with healthy ones from another person (a donor). This procedure allows the recipient to get new stem cells that work properly to restore the marrow function of patients who have had severe injury to that site. Marrow injury can occur because of primary marrow failure (e.g. sickle cell anemia),²⁹ destruction of marrow by disease (e.g. leukemia), or intensive chemical or radiation exposure.

A key step for the success of cellular therapy is to have the injected cells home to the injured site and be retained there to repair the damaged tissue. The cell trafficking is mainly controlled through the interaction of a cell surface receptor called CXCR4 with the chemotactic cytokine (a growth factor that attracts cells) stromal cell derived factor-1 (SDF-1)^{30,31}. SDF-1 binding to its receptor CXCR4 on the cell surface provides essential signals for mobilization and homing of target cells to the injured site.³²⁻³⁴ Disruption of SDF-1/CXCR4 interaction can impair the engraftment of progenitor cells into sites of ischemia and disturbed ischemic limb neovascularization.³⁵ The injured tissues secrete large amount of SDF-1 to attract the progenitor cells to repair the damage³⁶⁻³⁸. More CXCR4 surface expression has been shown to increase the efficiency of cell homing and the resulting therapeutic effect^{39,40}.

Previous methods to increase CXCR4 expression include the culture of cells with medium containing serum⁴¹, or gene transfer⁴². These methods can take a long time to work, and are not currently practical for clinical use. In contrast, the method of the invention provides an efficient and convenient way to upregulate surface CXCR4 expression, and hence improve cell homing and engraftment into target tissue. The method of the invention has the advantages of short time processing (<4 hours) and no requirement for exogenous protein addition.

### SUMMARY OF THE INVENTION

This invention relates to a method and compositions for activation of precursor cells to have a better homing capability and higher viability. The methods involve incubation of the target cells with a designed medium (“invented medium” herein) at certain temperatures within a range of duration. Such activated cells have better ability to home to the target tissue to execute the therapeutic function.

The method includes the incubation of the target cells in the invented medium at 18-37° C. for less than 5 hours (1-4 hours). The incubation can be during or after the harvest of the progenitor cells. For example, bone marrow cells are harvested and centrifuged. The bone marrow cells in the pellet are resuspended in the invented solution and incubated for 2-4 hours. Bone marrow can also be harvested directly into the invented solution in higher concentration, or mixed with a solute powder to have a mixture with a final concentration that is the same as the invented solution. The mixed cell solution will then be processed for centrifugation to isolate the mononuclear cells. If the isolation process is shorter than 1 hour, the result cells may be suspended in the invented solution and incubated for a longer time (e.g. 2 hours) at 37° C.

The basic composition of the invented solution is a certain range of CaCl₂ or another calcium salt in a buffer solution. The buffer solution could be Phosphate Buffered Saline (PBS), or HEPES, or other buffer suitable for cell culture. In a preferred formulation, PBS (pH 7.0-7.2) is composed of about 1.54 mM potassium phosphate monobasic (KH₂PO₄), 2.71 mM sodium phosphate dibasic (Na₂HPO₄.7H₂O), and 155 mM sodium chloride (NaCl). Calcium in the invented solution is effective in a broad range. The best activation, with mouse tissues, is observed at a calcium concentration of about 0.3 mM (millimolar) to about 4 mM, and optimally in a range of about 0.5 mM to about 2 mM. Glucose is preferably included in the solution, at a concentration of about 1 mM to about 30 mM.

Additional components may be added into the invented solution during incubation. The additional components are small molecules with known composition and concentration. They include inorganic salts, amino acids, and vitamins. After the incubation, the cells can be directly used or washed with PBS once and then resuspended in PBS for therapeutic use.

Incubation of BMC with the invented solution at 37° C. for 4 hours significantly increased the surface expression of CXCR4 (FIG. 1), resulting in higher mobility of the cells towards SDF-1 (FIG. 2). When the treated BMC were intravenously injected into mice that had undergone ischemic surgery, more injected cells were detected in the ischemic muscles of the mice that received the treated BMC than those that received untreated BMC (FIG. 3). Better re-vascularization was observed in the ischemic mice injected with the treated BMC than the untreated group (FIG. 4). These data indicate that the invented treatment of BMC will increase surface CXCR4 expression, which results in the enhanced BMC homing to ischemic site. More BMC homing to the ischemic site promotes greater neo-vascularization and better therapeutic effect.

BMC treated with the invented method also homed significantly more into bone marrow than the untreated cells, when the BMC were injected into mice that had been irradiated to cause bone marrow damage (FIG. 5).

The invention can be used for cell based therapy for vascular diseases (Ischemia, atherosclerosis, and heart attack), wound healing, neural diseases, diabetes, cancer, immunotherapy, stem cell transplantation, and other uses. The invention is a method to activate progenitor cells that can be used to promote angiogenesis, wound healing, neural regeneration, and bone marrow replacement, and to inhibit atherosclerosis. Bone marrow cells or any target cells that are treated with the invented solution will home more efficiently to the damaged site and exert enhanced repair function. Patients with vascular diseases, who are undergoing cell-based therapy, will have improved outcomes. Less donor cells will be required for patients undergoing bone marrow or cord blood transplantation when the donor cells are treated with the invented method.

Currently, bone marrow cells or other progenitor cells for therapeutic use are either freshly isolated, or cultured in medium with serum. The freshly isolated cells are less efficient in response to chemokine signaling. The traditional culture process in medium with serum may increase the cell sensitivity to the signal, but the process is time consuming and risky (contamination, immunoreaction, extra manipulations). The invention provide a simple culture method using a simple buffered solution with known components (all small molecules, no proteins), and will not induce immune reaction when the treated cells are injected into body. The treatment time is only 1-4 hours. The therapeutic outcome is significantly better than the freshly isolated cells.

### BRIEF DESCRIPTION OF THE DRAWINGS

FIG. 1 shows a FACS analysis of surface CXCR4 expression on BMC, showing increased fluorescence indicative of increased CXCR4 expression when treated with the invented solution.

FIG. 2 shows BMC migration towards SDF-1 and its enhancement when treated with the inventive solution.

FIG. 3 shows improved homing of treated cells to an ischemic site.

FIG. 4 shows the effect of BMC injection on angiogenesis.

FIG. 5 shows increased homing of treated cells to bone marrow.

### DETAILED DESCRIPTION OF THE INVENTION

### Definitions

The term “cells” in the present invention refers to isolated mammalian tissue cells including, but not limited to, stem cells, progenitor cells, bone marrow-derived cells (BMC), and bone marrow-derived mononuclear cells (BM-MNC]. Other cell types may also be used in the methods of the invention, including endothelial progenitor cells (EPC), cord blood-derived cells, adipose derived cells, spleen-derived cells, hematopoietic stem cells, hematopoietic progenitor cells, mesenchymal stem cells (MSC), stromal cells, myocardial stem cells, myocardial cells, neuronal progenitor cells, precursor cells, mature cells, somatic cells, smooth muscle cells, fibroblasts, dendritic cells, astrocytes, and islet cells.

Cells can be treated as described in the invention so as to enhance the surface CXCR4 expression and improve the efficiency of engraftment. Cells which are treated with the invented method are referred to as treated cells. Cells can be obtained from the same individual human or animal into which the treated cells will be injected, or can be obtained from different individual. The treated cells will be used for therapy for diseases or for research in animals.

The term “Invented solution” refers to any buffered isotonic solution, containing calcium ions in a concentration that increases the concentration of CXCR4 surface receptors on target cells, in comparison to an equivalent solution that does not contain calcium. Additional components, in addition to saline, buffer and calcium ion, can include glucose, salt, amino acids, vitamins and similar well-known additives to cell culture solutions. A preferred pH range is a pH of about 7-7.5. Added glucose in the range of about 1 to about 30 mM is a preferred additive.

The term “invented treatment” refers to incubation of target cells in the invented solution (“treatment”) for certain times at certain temperature, for example, 2 hrs at 37° C. The incubation duration can be 0.5 hr to 15 hrs, preferably about 1 to about 4 hrs. The incubation temperature can be from 18° C. to 37° C. The treatment can be carried out prior to or during cell harvesting, cell processing, or cell injection.

The term “CXCR4” or “CXC Chemokine Receptor 4” refers to a protein with the property of binding with chemokine stromal cell-derived factor-1 (SDF-1). In particular, CXCR4 usually refers to the surface receptor for SDF-1.

### EXAMPLES

### 1. CXCR4 Surface Expression on BMC was Enhanced by the Invented Treatment

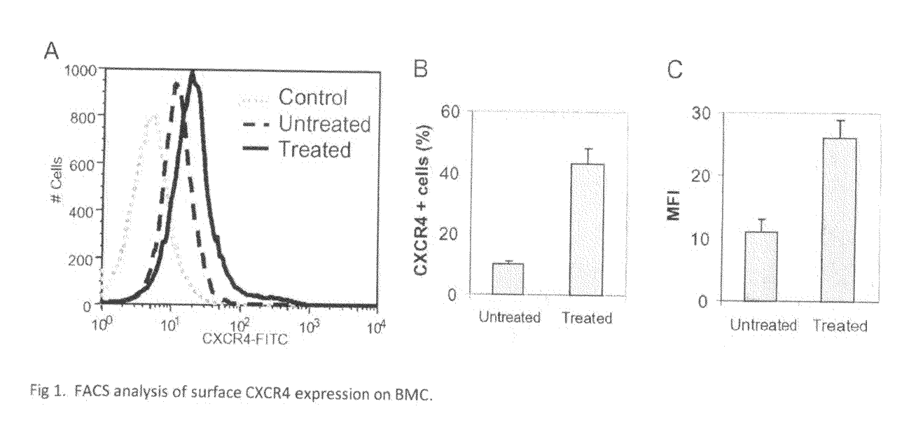

Mouse BM (bone marrow) was collected by flushing the femur with phosphate buffered saline (PBS). After centrifugation, the cells were suspended in 1.2 ml red blood cell lysis solution (Sigma) and incubated at 37° C. for 5 minutes followed by addition of 10 ml PBS and centrifugation to remove the lysed red blood cells. The BMC (bone marrow cells) numbering about 1×10⁶ cells were then incubated in PBS (“untreated”) or the invented solution (“treated”) for 4 hrs at 37° C. After a wash in phosphate-buffered saline (PBS), the BMC were re-suspended in 100 μl protein blocking solution with 20 fluorescent FITC-conjugated rat anti-(mouse CXCR4) monoclonal antibody (mAb) (BD-Pharmingen), and incubated at 4° C. for 1 hr. The fluorescence on the cell surface was measured by flow cytometry (FACS) using isotype IgG stained BMC as a control (control, grey line) to set the gate (FIG. 1A).

The results are shown in FIG. 1. There were significantly more cells expressing surface CXCR4 in the treated BMC (43±5%) than the untreated BMC (10±1%) (FIG. 1B). There was also significantly more surface CXCR4 on each treated cell than the untreated cell. The mean fluorescence intensity (MFI) increased from 11±2% untreated to 26±3% after the treatment (FIG. 1C).

### 2. BMC Mobility is Enhanced after the Invented Treatment

BMC migration towards SDF-1 was determined using a modified Boyden chamber assay. BMC were pre-incubated with either PBS or the invented solution for 4 hours at 37 deg.C. and transferred to the upper chamber of inserts in a 24-well plate containing DMEM and 100 ng/ml SDF-1. Migrated cells were counted after 6 hrs incubation at 37° C. Inhibitors AMD3100, LY294002, L-NMMA, and anti-CXCR4 antibody were added to the upper chamber along with BMC.

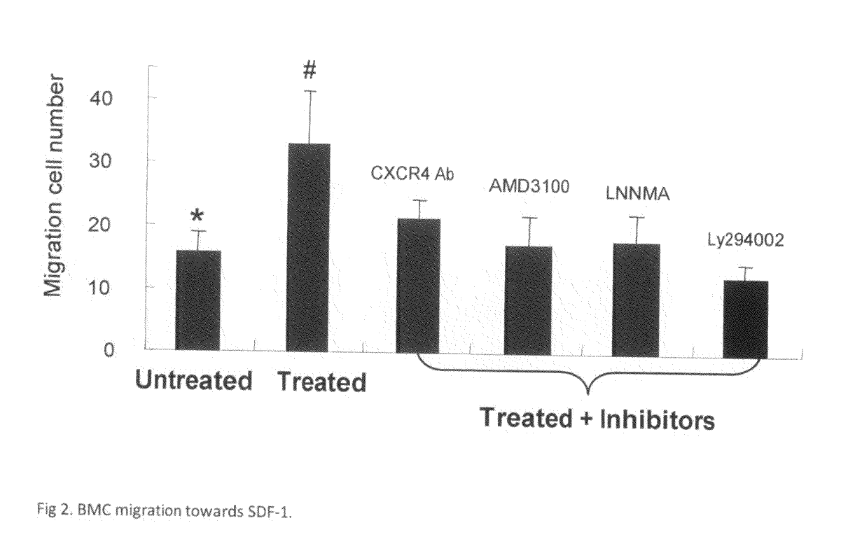

As shown in FIG. 2 (legend: *, P<0.05 versus treated group; #, P<0.05 versus other groups), cell mobility increased 2-fold after the invented treatment relative to untreated control (32.9±8.4 vs. 15.6±2.9; P<0.05, n=4, FIG. 2). The mobility of the treated BMC was significantly reduced by addition of PI-3K inhibitor LY294002, or nitric oxide synthase inhibitor L-NNMA, CXCR4 antagonist AMD3100, or anti-CXCR4 antibody (FIG. 2; all p<0.05). These results indicate that cell mobility is enhanced by the treatment, and that the enhancement is through SDF-1/CXCR4 interaction.

### 3. Treated BMC Home to the Injured Tissue Significantly Better

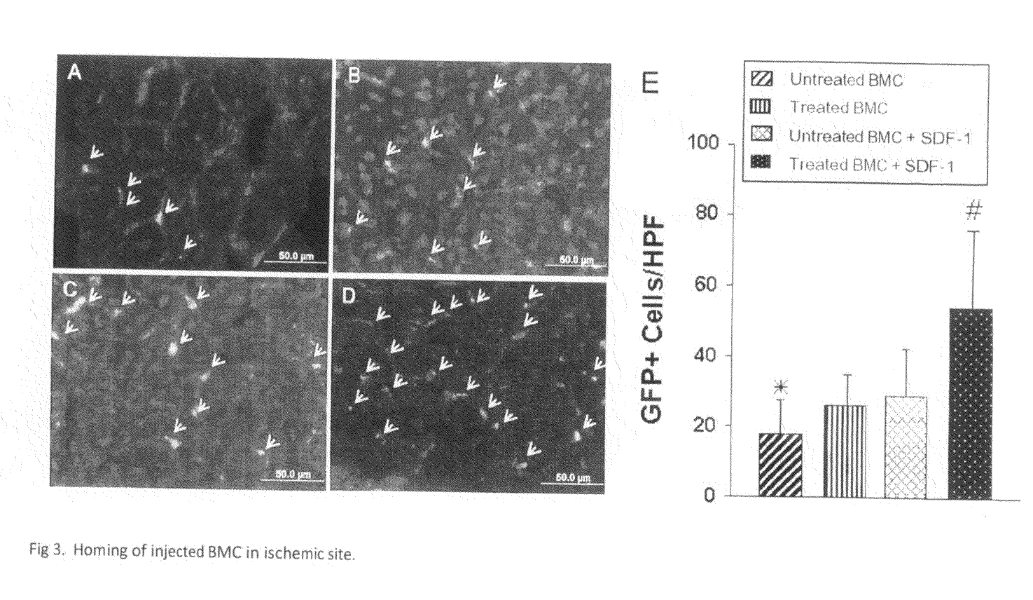

As shown in FIG. 3, BMC from GFP mice were incubated with the invented solution (treated) or with PBS (untreated), and injected into the tail veins of mice after surgical implementation of hindlimb ischemia. T

SDF1 protein was also injected into the ischemic hindlimb muscle to enhance BMC homing. SDF-1 protein (100 ng) was injected into the ischemic hindlimb muscle of WT BL6 mice twice at consecutive days after the surgery. The hindlimb muscles were recovered 7 days after the cell injection, stained with DAPI for nucleus (light white), and examined for GFP cells (bright white, arrow pointed) incorporation under fluorescent microscopy. (A) untreated BMC, B) Treated BMC, C) Untreated BMC+SDF-1, D) Treated BMC+SDF-1. E) The incorporated GFP cells were quantified as cells per high power field (HPF). *P<0.05, and **P<0.01 versus all other groups (n=6).

Significantly more injected GFP+ cells were detected in the ischemic muscles of the mice that received treated BMCs (FIG. 3B) than in mice that received untreated cells (FIG. 3A) in the absence of SDF-1. With exogenous SDF-1, both cells homed more efficiently (FIG. 3E). However, treated BMC (FIG. 3D) still homed more efficiently to the ischemic site than the control BMC (FIG. 3C).

### 4. Treated BMC Promotes Angiogenesis Significantly More Efficiently

BL6 mice with an ischemic hindlimb were injected intravenously with BMC that had been incubated in PBS (untreated) or the invented solution (treated) at 37° C. for 4 hours. SDF-1 protein was injected into the ischemic hindlimb muscle after the surgery. Blood flow of the lower limbs was measured using a laser Doppler perfusion image (LDPI) analyzer.

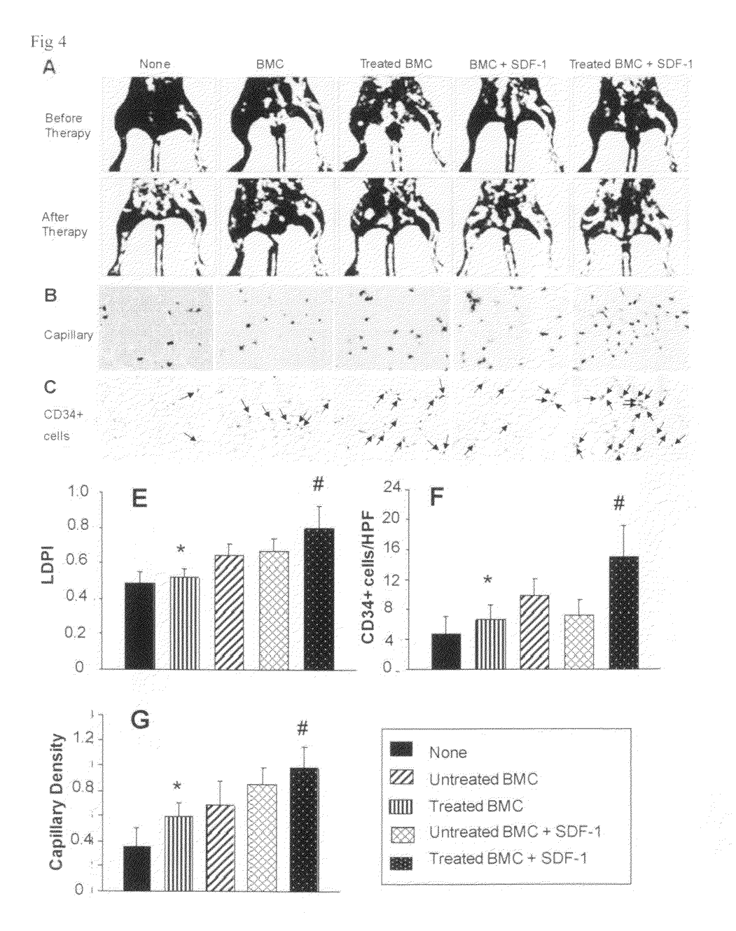

FIG. 4 shows the results. A). Representative laser Doppler perfusion images at day 0 and day 21 after ischemia and cell injection. B) Capillaries in the ischemic muscle from the mice at day 21 were identified by alkaline phosphatase cryosectional staining. C). CD34 immunostaining of paraffin sections obtained from the ischemic muscle of mice at day 7. Arrow points CD34+ cells. Dark dots represents cells counter-stained with Hemotoxylin. D). Quantitative measurement of perfusion ratio of ischemic limbs to that of normal limbs at day 21 (n=6). E) Quantification of capillary density on the tissue section, which is presented as the ratio of the number of capillaries to the number of muscle fibers. F), Quantification of CD34+ cells around each muscle fiber. *P<0.05 versus Treated group. #P<0.05 versus other groups.

It can thus be seen that injection of the treated BMC promoted significantly improved reperfusion (FIGS. 4A and D) and higher capillary density (FIGS. 4B and E) in ischemic limbs at 3 weeks after surgery both in the presence and absence, of SDF-1 injections. The improved therapy was paralleled by significantly more CD34+ cells in the ischemic muscles following injections of the treated BMC (FIGS. 4C and F).

### 5. The Invented Treatment Enhanced BMC Homing to the BM of Irradiated Mice

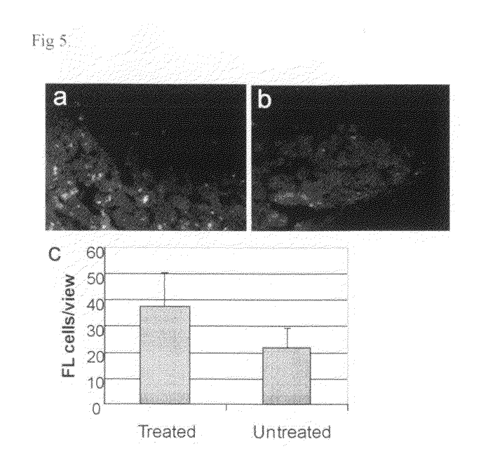

BMC (1×10 exp 7) isolated from mice were treated with a) the invented method or b) PBS (untreated), and then were intravenously injected into recipient mice that had received total body irradiation (550 cGy) one day before cell injection. The mice were sacrificed 3 days later, and sections of the recovered bones were observed under fluorescence microscopy. As shown in FIG. 5, (a) shows cells injected with the treated BMC, and (b) shows cells injected with untreated BMC. The homed cells with red fluorescence were quantified as cells per view under 20× objective lens (c). Significantly more BMCs treated with the invented method homed into bone marrow than the untreated cells.

Hence, it has been discovered that treatment of BMCs with the inventive solution improves the ability of the BMCs to repair damage. The treated BMC, relative to control, have increased CXCR4 expression, improved cell mobility, improved homing to the tissue, and improved angiogenesis induction.

The treatment of the invention is an ideal treatment in terms of minimization of adverse effect potential. The treatment is accomplished by adding an effective level of calcium to BMC cells in a PBS solution and incubating for 1-4 hours before injection of the cells into a target site, without necessarily removing the incubation solution. The total quantity of calcium injected during treatment is very small and unlikely to affect any other physiological process. Addition of low levels of glucose can further improve the response.

While treatment of animals with this procedure is of interest, the important aspect of this invention is the treatment of humans. It is likely that humans—and for that matter mammals other than mice—may have optimal response to this treatment system at varying levels of calcium concentration, and perhaps of other variables. Accordingly, the invention is in an important aspect the use of optimal levels of calcium in the treatment of bone marrow cells to make them clinically useful in the restoration of one or more functions. Determination of the optimum concentration is straightforward for any species once the utility is known. In particular, it is important for use in humans to determine experimentally. for the particular cells involved in the treatment, the optimal value of one or more of the concentration of calcium ions, the time of incubation, and the temperature of incubation, to allow for standardization of clinical procedures.

### REFERENCES CITED

1. Tateishi-Yuyama E, Matsubara H, Murohara T, et al. Therapeutic angiogenesis for patients with limb ischaemia by autologous transplantation of bone-marrow cells: a pilot study and a randomized controlled trial. The Lancet. 2002; 360:427-435.
2. Gersh B J, Simari R D. Cardiac stem cell repair therapy: a clinical perspective. Indian Heart J. 2006; 58:308-314.
3. Liu Z-J, Velazquez O C. Hyperoxia, Endothelial Progenitor Cell Mobilization, and Diabetic Wound Healing. Antioxidants & Redox Signaling 2008; 10:1869-1882.
4. Rehni A K, Singh I, Singh N, Kumar M. Stem cells: implications in experimental ischaemic stroke therapy. Stem Cell Rev. 2008; 4:227-233.
5. Baker P S, Brown G C. Stem-cell therapy in retinal disease. Curr Opin Ophthalmol. 2009; 20:175-181.
6. Meuth S G, Kleinschnitz C, Wiendl H. Recent clinical trials and future therapies. J Neurol. 2008; 255 Suppl 6:93-96.
7. Bensinger W I. Role of autologous and allogeneic stem cell transplantation in myeloma. Leukemia. 2009; 23:442-448.
8. Higashi Y, Kimura M, Hara K, et al. Autologous bone-marrow mononuclear cell implantation improves endothelium-dependent vasodilation in patients with limb ischemia. Circulation. 2004; 109:1215-1218.
9. Miyamoto M, Yasutake M, Takano H, et al. Therapeutic angiogenesis by autologous bone marrow cell implantation for refractory chronic peripheral arterial disease using assessment of neovascularization by 99 mTc-tetrofosmin (TF) perfusion scintigraphy. Cell Transplant. 2004; 13:429-437.
10. Asahara T, Murohara T, Sullivan A, et al. Isolation of Putative Progenitor Endothelial Cells for Angiogenesis. Science. 1997; 275:964-966.
11. Murohara T, Ikeda H, Duan J, et al. Transplanted cord blood-derived endothelial precursor cells augment postnatal neovascularization. J Clin Invest. 2000; 105:1527-1536.
12. Huang P, Li 5, Han M, Xiao Z, Yang R, Han Z C. Autologous Transplantation of Granulocyte Colony-Stimulating Factor-Mobilized Peripheral Blood Mononuclear Cells Improves Critical Limb Ischemia in Diabetes. Diabetes Care. 2005; 28:2155-2160.
13. Tateno K, Minamino T, Toko H, et al. Critical roles of muscle-secreted angiogenic factors in therapeutic neovascularization. Circ Res. 2006; 98:1194-1202.
14. Luttun A, Tjwa M, Moons L, et al. Revascularization of ischemic tissues by PlGF treatment, and inhibition of tumor angiogenesis, arthritis and atherosclerosis by anti-Flt1. Nat Med. 2002; 8:831-840.
15. Rauscher F M, Goldschmidt-Clermont P J, Davis B H, et al. Aging, Progenitor Cell Exhaustion, and Atherosclerosis. Circulation. 2003; 108:457-463.
16. Sata M, Saiura A, Kunisato A, et al. Hematopoietic stem cells differentiate into vascular cells that participate in the pathogenesis of atherosclerosis. Nat Med. 2002; 8:403-409.
17. George J, Afek A, Abashidze A, et al. Transfer of endothelial progenitor and bone marrow cells influences atherosclerotic plaque size and composition in apolipoprotein E knockout mice. Arterioscler Thromb Vasc Biol. 2005; 25:2636-2641.
18. Werner N, Nickenig G. Endothelial progenitor cells in health and atherosclerotic disease. Ann Med. 2007; 39:82-90.
19. Masuda H, Asahara T. Post-natal endothelial progenitor cells for neovascularization in tissue regeneration. Cardiovasc Res. 2003; 58:390-398.
20. Purhonen S, Palm J, Rossi D, et al. Bone marrow-derived circulating endothelial precursors do not contribute to vascular endothelium and are not needed for tumor growth. Proc Natl Acad Sci USA. 2008; 105:6620-6625.
21. Kalka C, Masuda H, Takahashi T, et al. Transplantation of ex vivo expanded endothelial progenitor cells for therapeutic neovascularization. PNAS. 2000; 97:3422-3427.
22. Shintani S, Murohara T, Ikeda H, et al. Augmentation of postnatal neovascularization with autologous bone marrow transplantation. Circulation. 2001; 103:897-903.
23. Finney M R, Greco N J, Haynesworth S E, et al. Direct comparison of umbilical cord blood versus bone marrow-derived endothelial precursor cells in mediating neovascularization in response to vascular ischemia. Biol Blood Marrow Transplant. 2006; 12:585-593.
24. Kamihata H, Matsubara H, Nishiue T, et al. Implantation of bone marrow mononuclear cells into ischemic myocardium enhances collateral perfusion and regional function via side supply of angioblasts, angiogenic ligands, and cytokines. Circulation. 2001; 104:1046-1052.
25. Kocher A A, Schuster M D, Szabolcs M J, et al. Neovascularization of ischemic myocardium by human bone-marrow-derived angioblasts prevents cardiomyocyte apoptosis, reduces remodeling and improves cardiac function. Nat Med. 2001; 7:430-436.
26. Minamino K, Adachi Y, Okigaki M, et al. Macrophage Colony-Stimulating Factor (M-CSF), As Well As Granulocyte Colony-Stimulating Factor (G-CSF), Accelerates Neovascularization. Stem Cells. 2005; 23:347-354.
27. Ripa R S, Jorgensen E, Wang Y, et al. Stem cell mobilization induced by subcutaneous granulocyte-colony stimulating factor to improve cardiac regeneration after acute ST-elevation myocardial infarction: result of the double-blind, randomized, placebo-controlled stem cells in myocardial infarction (STEMMI) trial. Circulation. 2006; 113:1983-1992.
28. Zohlnhofer D, Ott I, Mehilli J, et al. Stem cell mobilization by granulocyte colony-stimulating factor in patients with acute myocardial infarction: a randomized controlled trial. Jama. 2006; 295:1003-1010.
29. Hsieh M M, Kang E M, Fitzhugh C D, et al. Allogeneic Hematopoietic Stem-Cell Transplantation for Sickle Cell Disease. N Engl J Med 2009; 361:2309-2317.
30. Grunewald M, Avraham I, Dor Y, et al. VEGF-Induced Adult Neovascularization: Recruitment, Retention, and Role of Accessory Cells. Cell. 2006; 124:175-189.
31. Yamaguchi J, Kusano K F, Masuo O, et al. Stromal cell-derived factor-1 effects on ex vivo expanded endothelial progenitor cell recruitment for ischemic neovascularization. Circulation. 2003; 107:1322-1328.
32. Lapidot T. Mechanism of human stem cell migration and repopulation of NOD/SCID and B2mnull NOD/SCID mice. The role of SDF-1/CXCR4 interactions. Ann NY Acad Sci. 2001; 938:83-95.
33. Wright D E, Bowman E P, Wagers A J, Butcher E C, Weissman I L. Hematopoietic stem cells are uniquely selective in their migratory response to chemokines. J Exp Med. 2002; 195:1145-1154.
34. De Falco E, Porcelli D, Torella A R, et al. SDF-1 involvement in endothelial phenotype and ischemia-induced recruitment of bone marrow progenitor cells. Blood. 2004; 104:3472-3482.
35. Imitola J, Raddassi K, Park K I, et al. Directed migration of neural stem cells to sites of CNS injury by the stromal cell-derived factor 1alpha/CXC chemokine receptor 4 pathway. Proc Natl Acad Sci USA. 2004; 101:18117-18122.
36. Askari A T, Unzek S, Popovic Z B, et al. Effect of stromal-cell-derived factor 1 on stem-cell homing and tissue regeneration in ischaemic cardiomyopathy. The Lancet. 2003; 362:697-703.
37. Ceradini D J, Kulkarni A R, Callaghan M J, et al. Progenitor cell trafficking is regulated by hypoxic gradients through HIF-1 induction of SDF-1. 2004; 10:858-864.
38. van Weel V, Seghers L, van Hinsbergh V, van Bockel H, Quax P. Upregulation of SDF1 and CXCR4 in ischemic tissue of patients with severe peripheral arterial disease. Vasc Pharmacol. 2006; 45:e62-e62.
39. Bhakta S, Hong P, Koc O. The surface adhesion molecule CXCR4 stimulates mesenchymal stem cell migration to stromal cell-derived factor-1 in vitro but does not decrease apoptosis under serum deprivation. Cardiovasc Revasc Med. 2006; 7:19-24.
40. Kahn J, Byk T, Jansson-Sjostrand L, et al. Overexpression of CXCR4 on human CD34+ progenitors increases their proliferation, migration, and NOD/SCID repopulation. Blood. 2004; 103:2942-2949.
41. Shiba Y, Takahashi M, Hata T, et al. Bone marrow CXCR4 induction by cultivation enhances therapeutic angiogenesis. Cardiovasc Res. 2009; 81:169-177.
42. Zhang D, Fan G-C, Zhou X, et al. Over-expression of CXCR4 on mesenchymal stem cells augments myoangiogenesis in the infarcted myocardium. J Mol Cell Cardio. 2008; 44:281-292.
43. Wu Q Shao H, Eton D, et al. Extracellular calcium increases CXCR4 Expression on Bone Marrow-derived Cells and Enhances Pro-Angiogenesis Therapy. J Cell Mol Med. 2009; 13(9B):3764-3773. (Note that reference 43 is a publication by the inventor).

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference, where such incorporation is permitted. Nothing herein is to be construed as an admission that the invention is not entitled to antedate such disclosure by virtue of prior invention, where relevant. Those skilled in the art will recognize, or be able to ascertain using no more than routine experimentation, many equivalents to the specific embodiments of the invention described herein. Such equivalents are intended to be encompassed by the following claims.

What is claimed is:

1. A method of activating one or more mammalian cells selected from progenitor cells, stem cells, and precursor cells to increase the concentration of the CXCR4 receptor on the surfaces of said cells, the method comprising:
suspending said cells in a buffered isotonic solution containing an effective amount of calcium ions;
incubating said cells in said solution for a period of about 1 hour or more; and observing an increase in the concentration of CXCR4 receptor.

2. A method according to claim 1 wherein the temperature of incubation is in the range of about 18 to about 37 deg. C.

3. The method of claim 1 for use with murine cells wherein the time of incubation is in the range of about 1 to about 4 hours.

4. The method of claim 1 for use with human cells in which the optimal time of incubation is determined experimentally.

5. The method of claim 1 in which the concentration of calcium ions is in the range of about 0.3 to about 5 mM.

6. The method of claim 1 for use with human cells in which the optimal concentration of calcium ions is determined experimentally.

7. The method of claim 1 further comprising glucose in a concentration of about 1 mM to about 30 mM.

8. The method of claim 1 for use with human cells in which the optimal concentration of glucose is determined experimentally.

9. The method of claim 1 for use with human cells, including, but not limited to, stem cells, progenitor cells, bone marrow-derived cells (BMC), and bone marrow-derived mononuclear cells (BM-MNC]. Other cell types may also be used in the methods of the invention, including endothelial progenitor cells (EPC), cord blood-derived cells, adipose derived cells, spleen-derived cells, hematopoietic stem cells, hematopoietic progenitor cells, mesenchymal stem cells (MSC), stromal cells, myocardial stem cells, myocardial cells, neuronal progenitor cells, precursor cells, mature cells, somatic cells, smooth muscle cells, fibroblasts, dendritic cells, astrocytes, and islet cells.

10. A method of enhancement of the outcome of a procedure by the incubation of cells in a buffered isotonic solution containing an effective amount of calcium ion,
wherein the enhancement is at least one of:
the homing of progenitor cells to an ischemic site;
the homing of therapeutic cells to target tissues;
the survival of transplanted cells at target tissue;
the efficiency of cell transplantation;
the homing of progenitor cells to an SDF-1 producing site;
the engraftment of progenitor cells to a target site; and
the promotion of at least one of neo-vascularization, angiogenesis, neuron-regeneration, and hematopoietic cell production.

11. The method of claim 10 in which one or more of the optimal concentration of calcium ions, the optimal time of incubation, and the optimal temperature of incubation is determined experimentally.

12. The method of claim 10 wherein the buffered isotonic solution further contains glucose

13. The method of claim 10 wherein the cells that are incubated are of human origin.

14. A method of enhancing the homing of selected cells to a selected target site, the method comprising:
suspending said cells in a buffered isotonic solution containing an effective amount of calcium ions; and incubating said cells in said solution for a period of about 1 hour or more, followed by injecting the cells in a location allowing them to migrate to the selected target site.

15. The method according to claim 14 wherein the temperature of incubation is in the range of about 18 to about 37 deg. C.

16. The method according to claim 14 for use with murine cells wherein the time of incubation is in the range of about 1 to about 4 hours.

17. The method according to claim 14 for use with human cells in which the optimal time of incubation is determined experimentally.

18. The method according to claim 14 in which the concentration of calcium ions is in the range of about 0.3 to about 5 mM

19. The method according to claim 14 for use with human cells in which one or more of the optimal concentration of calcium ions, the optimal time of incubation, and the optimal temperature of incubation is determined experimentally.

20. The method of claim 14 for use in humans, wherein said enhancement of homing is effective in treatment of a disease.
